(19) United States
(12) Patent Application Publication
(10) Pub. No.: US 2017/0327821 A1
(43) Pub. Date: Nov. 16, 2017

(54) OLIGONUCLEOTIDES FOR GENOMIC DNA EDITING

(71) Applicant: ProQR Therapeutics II B.V., Leiden (NL)

(72) Inventors: Maarten Holkers, Valkenburg ZH (NL); Jim Swildens, Leiden (NL)

(73) Assignee: ProQR Therapeutics II B.V., Leiden (NL)

(21) Appl. No.: 15/519,968

(22) PCT Filed: Oct. 23, 2015
(86) PCT No.: PCT/EP2015/074675

§ 371 Date: Apr. 18, 2017

(30) Foreign Application Priority Data
Oct. 23, 2014 (GB) 1418892.4

(51) Int. Cl. C12N 15/11; C12N 15/113; A61K 31/7088; C12N 15/10

(52) U.S. Cl. CPC C12N 15/111; C12N 15/102; C12N 15/1138; A61K 31/7088; C12N 2310/323; C12N 2320/30; C12N 2310/315

(57) ABSTRACT

A method for making a change in an endogenous chromosomal DNA sequence of a mammalian cell, comprising steps of: (i) introducing into said cell an oligonucleotide having a sequence that is complementary to the chromosomal DNA sequence and that includes the change; (ii) allowing sufficient time for the cell to incorporate the change into the endogenous chromosomal DNA sequence through endogenous nucleic acid modifying pathways; and (iii) identifying the presence of the change in the chromosomal DNA sequence. The invention is particularly useful for correcting mutations in the CFTR gene.

This application claims the benefit of United Kingdom patent application 1418892.4, the complete contents of which are hereby incorporated herein by reference for all purposes.

### TECHNICAL FIELD

The invention is in the field of gene editing, whereby the nucleotide sequence of a chromosomal DNA locus is modified e.g. to correct a mutation.

### BACKGROUND ART

The use of oligonucleotides to edit genomic DNA is known in the art e.g. see Papaioannou et al. (Expert Opin Biol Ther. 2012 Mar; 12(3):329-42).

Aarts & Riele (Nucleic Acids Research 2010, 38:20) disclose methods using single-stranded DNA oligonucleotides (ssODNs) for correcting the sequence and restoring the function of a mutant fluorescent reporter gene which had been integrated into the genome of mouse ES cells. They report that unmodified ssODNs gave better results than those having end-protected phosphorothioate linkages. The same authors have reported further similar results (Aarts & Riele, J Cell Mol Med 2010, 14(6B):1657-67).

Andrieu-Soler et al. (Nucleic Acids Research 2005, 33:3833-3742) used single-stranded DNAs with or without various forms of end-protection to modify a fluorescent reporter gene in 293T cells. They saw the best results using 25mer oligonucleotides having flanking locked nucleic acid (LNA) residues or with terminal phosphorothioate linkages. They also tested the LNA-flanked oligonucleotides in vivo in mice and reported a phenotypic improvement, but they did not check the genome sequence of these cells to confirm that sequence modification had occurred. In later work, however, oligonucleotides with terminal phosphorothioate linkages were shown to function in mice in vivo (Andrieu-Soler et al., Molecular Vision 2007, 13:692-706).

Papaioannou et al. (J Gene Med. 2009 11(3):267-74) report that internally-protected ssODNs using phosphorothioate linkages gave better results in CHO cells than end-protected ssODNs. They also used siRNA to suppress endogenous MSH2 expression.

Another DNA editing technique which uses oligonucleotides is known as CRISPR, but this technique requires co-delivery of the CRISPR/Cas9 enzyme together with the oligonucleotide.

There remains a need for new techniques which can utilise endogenous cellular pathways to edit endogenous genes in mammalian cells, even in whole organisms.

### DISCLOSURE OF THE INVENTION

The invention provides a method for making a change in an endogenous chromosomal DNA sequence of a mammalian cell, comprising steps of: (i) introducing into said cell an oligonucleotide having a sequence that is complementary to the chromosomal DNA sequence except for the change; (ii) allowing sufficient time for the cell to incorporate the change into the endogenous chromosomal DNA sequence through endogenous nucleic acid modifying pathways; and (iii) identifying the presence of the change in the chromosomal DNA sequence.

The invention also provides a method for making a change in an endogenous mutant CFTR chromosomal DNA sequence of a human cell, comprising steps of: (i) introducing into said cell an oligonucleotide having a sequence that is complementary to the chromosomal DNA sequence except for the change; and (ii) allowing sufficient time for the cell to incorporate the change into the endogenous chromosomal DNA sequence through endogenous nucleic acid modifying pathways.

The invention also provides an oligonucleotide having a sequence that is complementary to a target sequence in an endogenous mammalian chromosomal DNA sequence, except that it includes a desired modification of the target sequence, wherein the oligonucleotide has one or more of the following structural features: (i) it includes at least one locked nucleoside; (ii) it includes at least one phosphorothioate inter-nucleotide linkage; and/or (iii) it is at least 26 nucleotides long e.g. 45-100 nucleotides long.

The invention also provides an oligonucleotide having a sequence that is complementary to a target sequence in an endogenous mammalian chromosomal DNA sequence, except that it includes at an internal position a desired modification of the target sequence, wherein the nucleotide immediately upstream of the internal position is a locked nucleotide. Preferably the two nucleotides immediately upstream of the internal position are both locked.

The invention also provides an oligonucleotide having a sequence that is complementary to a target sequence in an endogenous mammalian chromosomal DNA sequence, except that it includes at an internal position a desired modification of the target sequence, wherein the nucleotide immediately upstream of the internal position and/or the nucleotide immediately downstream of the internal position is linked to a nucleotide at the internal position via a phosphorothioate linkage.

The invention also provides an oligonucleotide for making a desired insertion or substitution at a specific position in a chosen strand of a target chromosomal DNA sequence, having sequence 5′-X-Y-Z-3′, wherein: X is complementary to the chromosomal sequence downstream in the non-chosen strand of the specific position; Z is complementary to the chromosomal sequence upstream in the non-chosen strand of the specific position; and Y is the desired insertion or substitution. The chosen strand is preferably the antisense strand, and so the non-chosen strand is the sense strand. X and/or Z may be linked to Y by a phosphorothioate linkage.

The invention also provides an oligonucleotide having a sequence that is complementary to a target sequence in an endogenous mammalian chromosomal DNA sequence, except that it includes a desired modification of the target sequence, wherein (i) at least the 5′ and/or 3′ terminal nucleotides are locked; and/or (ii) at least the 5′ and/or 3′ terminal dinucleotides are linked via a phosphorothioate linkage.

The invention also provides an oligonucleotide having a sequence that is complementary to a target sequence in an endogenous mammalian chromosomal DNA sequence, except that it includes at an internal position a desired modification of the target sequence, wherein a nucleotide immediately downstream and/or upstream of the internal position is (i) linked to a nucleotide at the internal position via a phosphorothioate linkage and/or (ii) a locked nucleotide. Further nucleotides downstream and/or upstream may be similarly modified, such that two or more consecutive nucleotides can have properties (i) and/or (ii).

Similarly, the invention also provides an oligonucleotide for making a desired insertion or substitution at a specific position in a chosen strand of a target chromosomal DNA sequence, having sequence 5′-X-Y-Z-3′, wherein: X is complementary to the chromosomal sequence downstream in the non-chosen strand of the specific position; Z is complementary to the chromosomal sequence upstream in the non-chosen strand of the specific position; and Y is the desired insertion or substitution; and wherein (i) X and/or Z is/are linked to Y by a phosphorothioate linkage, and/or (ii) the 3′ nucleotide of X and/or the 5′ nucleotide of Z is a locked nucleotide. Further details of these oligonucleotides are discussed below.

The invention also provides an oligonucleotide comprising the nucleotide sequence of any one of SEQ ID NOs: 1 to 6 or of any one of SEQ ID NOs: 7 to 10.

The invention also provides an oligonucleotide for correcting the ΔF508 CFTR mutation, having sequence 5′-X-Y-Z-3′, wherein: X is complementary to the sense strand of the human CFTR gene, starting at nucleotide 1524; Z is complementary to the sense strand of the human CFTR gene, up to nucleotide 1520; and Y is a trinucleotide AAG, AAA or AAT. The human CFTR nucleotide numbering herein is standard and is based on the sense strand (SEQ ID NO: 11). It is also possible to restore CFTR function without restoring a Phe codon, as amino acids Met, Gly and Cys can also function at position 508. Thus in such embodiments sequence Y can alternatively be CCG, CAG, CCA, CAA, CCT or CAT.

The invention also provides an oligonucleotide for correcting the ΔF508 CFTR mutation, having sequence 5′-X-Y-Z-3′, wherein: X is complementary to the antisense strand of the human CFTR gene, up to nucleotide 1520; Z is complementary to the antisense strand of the human CFTR gene, starting at nucleotide 1524; and Y is a trinucleotide CTT, TTT or ATT. As mentioned above, it is also possible to restore CFTR function without restoring a Phe codon, so sequence Y can alternatively be CGG, CTG, TGG, TTG, AGG or ATG.

The invention similarly provides an oligonucleotide for correcting the ΔF508 CFTR mutation, having sequence 5′-X-Y-Z-3′, wherein: X is complementary to the sense strand of the human CFTR gene, starting at nucleotide 1525; Z is complementary to the sense strand of the human CFTR gene, up to nucleotide 1521; and Y is a trinucleotide AAA or GAA. As mentioned above, it is also possible to restore CFTR function without restoring a Phe codon, so sequence Y can alternatively be ACC, GCC, TCC, CCC, ACA, GCA, or CAT.

The invention similarly provides an oligonucleotide for correcting the ΔF508 CFTR mutation, having sequence 5′-X-Y-Z-3′, wherein: X is complementary to the antisense strand of the human CFTR gene, up to nucleotide 1521; Z is complementary to the antisense strand of the human CFTR gene, starting at nucleotide 1525; and Y is a trinucleotide TTT or TTC. As mentioned above, it is also possible to restore CFTR function without restoring a Phe codon, so sequence Y can alternatively be GGT, GGC, GGA, GGG, TGT, TGC, or ATG.

The invention also provides an oligonucleotide comprising nucleotide sequence SEQ ID NO: 4. This oligonucleotide (preferably DNA) can comprise one or more PS-linkages and/or one or more locked nucleosides. Having at least one PS-linkage (e.g. two or more, such as three, consecutive linkages) directly flanking (e.g. directly upstream of) the AAG trinucleotide at positions 23-25 of SEQ ID NO: 4 is preferred (e.g. including the motif CA*AAG, CC*A*AAG, AC*C*A*AAG, AAG*AT, AAG*A*TG, AAG*A*T*GA, CA*AAG*AT, CC*A*AAG*A*TG, etc.). The overall length of the oligonucleotide can be 47 nt (i.e. SEQ ID NO: 4 alone), or it can be extended upstream and/or downstream e.g. to reach up to 80 nucleotides long.

The invention also provides a cell comprising an oligonucleotide of the invention.

The Mammalian Cell

The invention concerns the modification of chromosomal DNA sequences in mammalian cells. In principle the invention can be used with cells from any mammalian species, but it is preferably used with cells from a primate, and most preferably with a human cell.

The invention can be used with cells from any organ e.g. skin, lung, heart, kidney, liver, eye, brain, blood. The invention is particularly suitable for modifying sequences in epithelial cells, more preferably in gut or lung epithelial cells.

The invention can also be used with mammalian cells which are not naturally present in an organism e.g. with a cell line or with an embryonic stem (ES) cell.

The invention can be used with various types of stem cell, including pluripotent stem cells, totipotent stem cells, embryonic stem cells, induced pluripotent stem cells, etc.

The cell can be located in vitro or in vivo. One advantage of the invention is that it can be used with cells in situ in a living organism, but it can also be used with cells in culture. One way of using the invention is to modify a chromosomal sequence in a cell which has been removed from a patient, and which is then reintroduced to the patient after being modified according to the invention.

The invention can also be used to edit the genome of cells within an organoid. Organoids can be thought of as three-dimensional in vitro-derived tissues but are driven using specific conditions to generate individual, isolated tissues (e.g. see Lancaster & Knoblich, Science 2014, vol. 345 no. 6194 1247125). In a therapeutic setting they are useful because they can be derived in vitro from a patient's cells, and the organoids can then be re-introduced to the patient as autologous material which is less likely to be rejected than a normal transplant. Thus, according to another preferred embodiment, the invention may be practised on organoids grown from tissue samples taken from a patient (e.g. from their gastrointestinal tract; see Sala et al. J Surg Res. 2009; 156(2):205-12, and also Sato et al. Gastroenterology 2011; 141:1762-72); upon gene repair in accordance with the invention, the organoids, or stem cells residing within the organoids, may be used to transplant back into the patient to ameliorate organ function.

The cell will generally have a genetic mutation before being modified according to the invention. The mutation may be heterozygous or homozygous. The invention will typically be used to modify point mutations or small deletions (e.g. up to 3 nucleotides). Genes containing mutations of particular interest are discussed below. In some embodiments, however, the invention is used in the opposite way by introducing a disease-associated mutation into a cell line or an animal e.g. in order to provide a useful research tool for the disease in question.

The most preferred cell type for use with the invention is a human lung cell having a mutation in the CFTR gene, such as a ΔF508 mutation.

The Target Sequence and the Change

The invention is used to make a change in an endogenous chromosomal DNA sequence, also referred to as the target sequence. As mentioned above, the target sequence will generally include a mutation, such as a point mutation or a small deletion (fewer than 10 nucleotides e.g. a missing codon), before being modified according to the invention.

The chromosome may be a mitochondrial chromosome, but is more usually a nuclear chromosome.

Genes containing mutations of particular interest include, but are not limited to, the CFTR gene (the cystic fibrosis transmembrane conductor receptor), dystrophin, huntingtin, neurofibromin 1, neurofibromin 2, the 8-globin chain of haemoglobin, CEP290 (centrosomal protein 290 kDa), the HEXA gene of the 8-hexosaminidase A, and any one of the Usher genes (e.g. USH2B encoding Usherin) responsible for a form of genetic blindness called Usher syndrome. The target sequence will be selected accordingly, and the oligonucleotide will include the desired modification in order to correct the mutation.

The change introduced according to the invention will generally be a single nucleotide substitution, a short insertion (e.g. up to 3 nucleotides), or a short deletion (e.g. up to 3 nucleotides). The invention is particularly useful for inserting one or more nucleotides (in particular, 2 or more, such as 3 e.g. a codon) into a target sequence.

Where the target sequence is in a coding sequence, it is possible to target either the sense strand or the antisense strand. The antisense strand is the DNA strand which is transcribed and is thus complementary to the RNA; conversely, the sense strand is the DNA strand which is not transcribed. Targeting the antisense strand is preferred because, in general, the inventors have observed a higher frequency of successful modification when targeting this strand.

As mentioned above, the most preferred target sequence is a mutant CFTR gene. The most common disease-causing CFTR mutation results in a ΔF508 polypeptide mutation and is caused by loss of nucleotides 1521-3, spanning codons 507-508, leading at the polypeptide level to loss of Phe-508. Thus the target sequence may be this region of the CFTR gene, and the oligonucleotide will lead to the introduction of three nucleotides to provide a codon for phenylalanine in the modified sequence (or, as mentioned above, for another amino acid which still provides a CFTR protein that can function as an ion channel and thus relieve cystic fibrosis). Thus the oligonucleotide can include triplet CTT, TTT, or ATT for inserting after the second nucleotide of codon 507 (if targeting the sense strand) or the complement thereof (if targeting the antisense strand) namely AAG, AAA, or AAT; alternatively, the oligonucleotide can include triplet TTT or TTC for inserting after the third nucleotide of codon 507 (if targeting the sense strand) or the complement thereof (if targeting the antisense strand) namely AAA or GAA.

In addition to ΔF508 those skilled in the art of CF mutations recognise that between 1000 and 2000 mutations are known in the CFTR gene, including R117H, G542X, G551D, R553X, W1282X, and N1303K. All such mutations are amenable to modification using the methods of the invention, and oligonucleotides can be designed accordingly.

The target sequence is endogenous to the mammalian cell. Thus the target sequence is not, for instance, a transgene or a marker gene which has been artificially introduced at some point in the cell's history, but rather is a gene that is naturally present in the cell (whether in mutant or non-mutant form).

The Oligonucleotide

The invention utilises oligonucleotides to modify a target sequence in a mammalian cell. These oligonucleotides are generally single-stranded and can have various structural features, as discussed in more detail below.

The oligonucleotide will generally be shorter than 100 nucleotides in length, and will generally have from 20-80 or 25-75 nucleotides, and preferably has from 27-50 or 45-70 nucleotides. The inventors have seen very good results using oligonucleotides with between 40-50 nucleotides and between 50-60 nucleotides.

Where the desired modification is an insertion or substitution then the oligonucleotide has sequence X-Y-Z where the two outermost sequences (X & Z) are complementary to the regions of the target sequence that flank the position of the desired modification. These two regions are ideally each at least 10 nucleotides long e.g. between 20-35 or 10-25 nucleotides, such as 12 or 22 or 26 or 28 nucleotides long each. They can differ in length, but it is preferred that they are approximately equal in length e.g. differing in length by no more than 5 nucleotides. The middle region (Y) includes the desired modification i.e. the sequence to be inserted into the target sequence, or the sequence substitution to be made (such as a trinucleotide to be inserted). Thus the Y sequence specifies the desired modification (i.e. insertion or substitution) to the target sequence.

Where the desired modification is a deletion then the oligonucleotide has sequence X-Z where X & Z are complementary to the regions of the target sequence that flank the position of the desired deletion, as discussed above. The target sequence includes a short sequence between these two regions, and the invention leads to deletion of this short sequence. Thus the absence of this short sequence in the oligonucleotide specifies the desired modification (i.e. deletion) to the target sequence.

Without wishing to be bound by theory, a possible model for the DNA editing according to the invention envisages in case of an insertion or a substitution that the oligonucleotide has a “bulge” caused by sequence Y (e.g. see FIG. 8 of Aarts & Riele, 2010), whereas in case the DNA editing comprises a deletion, the target sequence comprises a bulge corresponding to the change. It is believed that the oligonucleotide is incorporated into the nascent chain during the process of DNA replication, thereby introducing the change into the nascent strand. During subsequent rounds of replication the newly formed chain incorporating the change is used as a template for normal replication whereupon the change is copied to the next chain, and so forth.

For all types of modification, the oligonucleotide has a sequence that is complementary to the chromosomal DNA sequence except for the desired change. Thus, for insertions and substitutions, regions X and Z are complementary to the target sequence but region Y is not, because this sequence is absent or is substituted in the chromosome. Similarly, for a deletion regions X and Z are complementary to the target sequence but the chromosome additionally includes a region which is not complementary to the oligonucleotide, and this is the region to be deleted.

In general, the position in the oligonucleotide which encodes the desired change (e.g. the desired insertion) will have at least 5 upstream and downstream nucleotides. Thus, for example, regions X and Z will both be at least 5 nucleotides long. More typically, these upstream and downstream regions will be longer e.g. at least 8, 10, 12 nucleotides. The upstream and downstream regions may be the same length, or different lengths.

The regions of the oligonucleotide which are complementary to the chromosomal DNA sequence are most preferably 100% complementary so that, after being incorporated, no changes are made to the genome except at the desired position. Nevertheless, the method can still proceed even if these complementary regions have a mismatch at a small number of positions provided that complementarity is generally maintained and the oligonucleotide can still hybridise with the target sequence (albeit with lower efficiency). Having less than 100% complementarity can also occur when the target sequence includes a polymorphic variant of the sequence for which the complementary regions were designed. Any such mismatches should not introduce stop codons, cryptic splice sites, or other features that could influence the normal functioning of the gene during transcription, pre-mRNA splicing or translation.

It is also possible to use non-Watson/Crick base pairing in these regions e.g. to include inosine residue(s).

The oligonucleotide can be a DNA oligonucleotide, but will more typically include at least one non-naturally occurring nucleotide. Thus the sugar moieties, purines, pyrimidines and/or backbone can differ from natural DNA. An oligonucleotide made of a mixture of DNA nucleotides and modified nucleotides is most typical.

The oligonucleotide can include one or more locked nucleoside(s), and thus one or more locked nucleotide(s). The ribose sugar in these nucleosides includes a bridge (usually a methylene bridge) which connects the 2′ oxygen and the 4′ carbon, thereby locking the ribose in the 3′-endo confirmation. Locked nucleosides can include bicyclic sugar chemistries, typically a constrained ethyl. 2′,4′-constrained 2′-O-methoxyethyl (cMOE) and 2′-O-ethyl (cEt) bicyclic nucleotides can be used (Pallan et al. 2012, Chem Commun (Camb). 48:8195-7). The inventors have observed a high frequency of successful modification when using oligonucleotides including locked nucleosides. In particular, in some embodiments of the invention an oligonucleotide has a locked nucleoside at its 5′ or 3′ terminus, or preferably at both the 5′ and 3′ termini. In other embodiments, the oligonucleotide has a locked nucleoside immediately to the 5′ and/or 3′ of the portion which specifies the desired modification of the target sequence (e.g. where the desired modification is an insertion or a substitution, the 3′-most nucleotide in X and/or the 5′-most nucleotide in Z, as defined above, is/are locked). In other embodiments, at least one of the three nucleotides immediately to the 5′ and/or 3′ of the portion which specifies the desired modification of the target sequence is locked (e.g. where the desired modification is an insertion or a substitution, the 3′-most trinucleotide in X and/or the 5′-most trinucleotide in Z, as defined above, include(s) at least one locked nucleoside).

Where an oligonucleotide includes a locked nucleoside then it is preferred to include at least two. More preferably, at least two (e.g. 2 or 3) neighbouring nucleotides both are locked. Thus in one embodiment the 2 or 3 nucleotides at both ends of the oligonucleotide are locked. In another embodiment, where the desired modification is an insertion or substitution, the 2 or 3 nucleotides at the 3′ end of X (i.e. immediately upstream of a sequence to be inserted) are locked.

The oligonucleotide can include one or more phosphorothioate internucleotide linkage(s). Compared to the natural DNA phosphodiester linkage, a phosphorothioate linkage (PS) substitutes a sulphur atom for a non-bridging oxygen. The inventors have observed a high frequency of successful modification when using oligonucleotides including phosphorothioate linkages. In particular, in some embodiments of the invention an oligonucleotide has phosphorothioate linkages at its 5′ or 3′ terminus, or preferably at both the 5′ and 3′ termini. Where an oligonucleotide includes phosphorothioate linkages then it is preferred to include at least two. For example, the linkages between up to 5 nucleotides at both ends of the oligonucleotide can be PS-linkages e.g. the 3 linkages between the 4 nucleotides at both ends. Other non-phosphodiester linkages can be used similarly.

The 3′ end of the oligonucleotide preferably has a free —OH group. The 5′ end of the oligonucleotide may have a free phosphate or phosphorothioate group, but in many embodiments the 5′ end of the oligonucleotide will instead be an —OH group. Other chemical groups for the 3′ and 5′ ends may also be used, as known in the art.

Specific oligonucleotides of interest for targeting the CFTR ΔF508 mutation comprise or consist of any one of SEQ ID NOs: 1 to 6 or 7 to 10, with SEQ ID NOs: 3 and 4 being of particular interest. Modified oligonucleotides of particular interest are as follows:

| SEQ | |
|---|---|
| ID | |
| NO: | Modifications |
| 3 | 6 PS linkages, between the 4 terminal nucleotides at both ends |
| 4 | 6 PS linkages, between the 4 terminal nucleotides at both ends |
| 4 | 6 locked nucleotides: 3 at each end |
| 4 | 2 locked nucleotides (nucleotides 21 & 22) |
| 4 | 2 locked nucleotides (nucleotides 26 & 27) |
| 4 | 3 PS linkages, between nucleotides 20-21, 21-22, and 22-23 |
| 9 | 3 PS linkages, between nucleotides 22-23, 23-24, and 24-25 |
| 10 | 3 PS linkages, between nucleotides 24-25, 25-26, and 26-27 |

In one preferred embodiment, as mentioned above, the invention provides an oligonucleotide for making a desired insertion or substitution at a specific position in a chosen strand of a target chromosomal DNA sequence, having sequence 5′-X-Y-Z-3′, wherein: X is complementary to the chromosomal sequence downstream in the non-chosen strand of the specific position; Z is complementary to the chromosomal sequence upstream in the non-chosen strand of the specific position; and Y is the desired insertion or substitution; and wherein (i) X and/or Z is/are linked to Y by a phosphorothioate linkage, and/or (ii) the 3′ nucleotide of X and/or the 5′ nucleotide of Z is a locked nucleotide.

In these oligonucleotides, properties (i) and/or (ii) preferably are present not only at the junction of X-Y and/or Y-Z, but continue further. Thus: (a) the linkage between n nucleotides at the 3′ end of X may be linked by phosphorothioate linkages, followed by a phosphorothioate linkage between the 5′ end of X and the 3′ nucleotide of Y; (b) the linkage between n nucleotides at the 5′ end of Z may be linked by phosphorothioate linkages, preceded by a phosphorothioate linkage between the 3′ nucleotide of Y and the 5′ nucleotide of Z; (c) the n nucleotides at the 3′ end of X may be locked nucleotides; and/or (d) the n nucleotides at the 5′ end of Z may be locked nucleotides. The value of n can be 1, 2, 3, 4, or 5 (or more). For instance, n can be 2 or 3.

Although it is possible for these oligonucleotides to include both phosphorothioate linkages and locked nucleotides, usually only one such modification is present in a single oligonucleotide. Similarly, although it is possible for the phosphorothioate linkage(s) and/or locked nucleotides to be present at both the 5′ and 3′ ends of Y, usually it/they are present at only one end e.g. in X. Thus, for instance, the oligonucleotide X-Y-Z can have three phosphorothioate linkages between the three 3′ nucleotides of X and the 5′ nucleotide of Y.

The oligonucleotide will usually be 20-100 nucleotides long. The oligonucleotide is preferably at least 25 nucleotides long e.g. at least or exactly 40, 50, 55, 57, or 60 nucleotides long.

The oligonucleotide is preferably has deoxyribose sugars (modified, where appropriate, by a locked modification as discussed above).

Y is preferably an insertion, which can be 1 or more nucleotides e.g. 2-6 nucleotides, and preferably 2 or 3 nucleotides (e.g. a missing codon).

The chosen strand is preferably the antisense strand, and so the non-chosen strand is the sense strand.

Incorporation of the Change Into the Chromosome

Methods of the invention include a step in which the oligonucleotide is introduced into a cell, and then sufficient time is allowed to let the cell incorporate the change (which is present in the oligonucleotide) into its chromosomal DNA. The change is mediated by endogenous nucleic acid modifying pathways in the cell which interact with the oligonucleotide and effect the change. Thus the method contrasts with, for example, CRISPR-based techniques which also use an oligonucleotide to facilitate DNA editing, but which require expression in the cell of an additional non-endogenous enzyme.

By the term “nucleic acid modifying pathways” the inventors do not wish to limit themselves to any particular pathway; any subset or all of the pathways involved in DNA replication, DNA repair and the like that are endogenous to the cell are envisaged by this term.

The amount of time which is required to permit the cell to incorporate the change can vary with different cell types but can easily be assessed by simple trials. The trials are particularly straightforward when the modified sequence leads to an easily-detected phenotypic change.

One suitable trial technique involves delivering the oligonucleotide to a test organism and then taking biopsy samples at various time points thereafter. The sequence of the target DNA can be assessed in the biopsy sequence and the proportion of cells having the modification can easily be followed. After this trial has been performed once then the knowledge can be retained and future delivery can be performed without needing to take biopsy samples.

A method of the invention can thus include a step of identifying the presence of the desired change in the cell's chromosomal DNA sequence, thereby verifying that the genome sequence has been modified. This step will typically involve sequencing of the relevant part of the chromosomal DNA, as discussed above, and the sequence change can thus be easily verified.

It is possible that a cell might divide after the oligonucleotide has been introduced into it, but before the genetic change has occurred. In these circumstances the oligonucleotide can still be present in both daughter cells and so they can both be considered as the cell into which the oligonucleotide was introduced. Thus the cell whose gene is corrected can be the cell which received the oligonucleotide, but sometimes it can be progeny of that receiving cell.

Furthermore, even after DNA modification has occurred the modified cells can become diluted (e.g. see FIG. 8 of Aarts & Riele, 2010). Thus in practical therapeutic terms a method of the invention may involve repeated delivery of an oligonucleotide until enough cells have been modified to provide a tangible benefit to the patient.

Delivery of the Oligonucleotide

Oligonucleotides of the invention are particularly suitable for therapeutic use, and so the invention provides a pharmaceutical composition comprising an oligonucleotide of the invention and a pharmaceutically acceptable carrier. In some embodiments of the invention the pharmaceutically acceptable carrier can simply be a saline solution. This can usefully be isotonic or hypotonic, particularly for pulmonary delivery.

The invention also provides a delivery device (e.g. syringe, inhaler, nebuliser) which includes a pharmaceutical composition of the invention.

The invention also provides an oligonucleotide of the invention for use in a method for making a change in an endogenous chromosomal DNA sequence of a mammalian cell, as described herein. Similarly, the invention provides the use of an oligonucleotide of the invention in the manufacture of a medicament for making a change in an endogenous chromosomal DNA sequence of a mammalian cell, as described herein.

The invention is particularly suitable for treating genetic diseases, such as cystic fibrosis, albinism, alpha-1-antitrypsin deficiency, Alzheimer disease, Amyotrophic lateral sclerosis, Asthma, β-thalassemia, Cadasil syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidormylosis bullosa, Fabry disease, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Hurler Syndrome, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-esol related cancer, Parkinson's disease, Peutz-Jeghers Syndrome, Phenylketonuria, Pompe's disease, Primary Ciliary Disease, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, various forms of cancer (e.g. BRCA1 and 2 linked breast cancer and ovarian cancer), and the like.

In some embodiments the oligonucleotide can be delivered systemically, but it is more typical to deliver an oligonucleotide to cells in which the target sequence's phenotype is seen. For instance, mutations in CFTR cause cystic fibrosis which is primarily seen in lung epithelial tissue, so with a CFTR target sequence it is preferred to deliver the oligonucleotide specifically and directly to the lungs. This can be conveniently achieved by inhalation e.g. of a powder or aerosol, typically via the use of a nebuliser. Especially preferred are nebulizers that use a so-called vibrating mesh, including the PARI eFlow (Rapid) or the i-neb from Respironics. The inventors have found that inhaled use of oligonucleotides can lead to systemic distribution of the oligonucleotide and uptake by cells in the gut, liver, pancreas, kidney and salivary gland tissues, among others. It is therefore to be expected that inhaled delivery of oligonucleotides according to the invention can also target these cells efficiently, which in the case of CFTR gene targeting could lead to amelioration of gastrointestinal symptoms also associated with cystic fibrosis. For other target sequences, depending on the disease and/or the target organ, administration may be topical (e.g. on the skin), intradermal, subcutaneous, intramuscular, intravenous, oral, ocular injection, etc.

In many diseases the mucus layer shows an increased thickness, leading to a decreased absorption of medicines via the lung. One such a disease is chronical bronchitis, another example is cystic fibrosis. Various forms of mucus normalizers are available, such as DNAses, hypertonic saline or mannitol, which is commercially available under the name of Bronchitol. When mucus normalizers are used in combination with DNA repair oligonucleotides, such as the oligonucleotides according to the invention, they might increase the effectiveness of those medicines. Accordingly, administration of an oligonucleotide according to the invention to a subject, preferably a human subject is preferably combined with mucus normalizers, preferably those mucus normalizers described herein. In addition, administration of the oligonucleotides according to the invention can be combined with administration of small molecule for treatment of CF, such as potentiator compounds for example Kalydeco (ivacaftor; VX-770), or corrector compounds, for example VX-809 (Lumacaftor) and/or VX-661.

Alternatively, or in combination with the mucus normalizers, delivery in mucus penetrating particles or nanoparticles can be applied for efficient delivery of RNA repair molecules to epithelial cells of for example lung and intestine. Accordingly, administration of an oligonucleotide according to the invention to a subject, preferably a human subject, preferably uses delivery in mucus penetrating particles or nanoparticles.

Chronic and acute lung infections are often present in patients with diseases such as cystic fibrosis. Antibiotic treatments reduce bacterial infections and the symptoms of those such as mucus thickening and/or biofilm formation. The use of antibiotics in combination with oligonucleotides according to the invention could increase effectiveness of the DNA repair due to easier access of the target cells for the repair molecule. Accordingly, administration of an oligonucleotide according to the invention to a subject, preferably a human subject, is preferably combined with antibiotic treatment to reduce bacterial infections and the symptoms of those such as mucus thickening and/or biofilm formation. The antibiotics can be administered systemically or locally or both.

For application in for example cystic fibrosis patients the oligonucleotides according to the invention, or packaged or complexed oligonucleotides according to the invention may be combined with any mucus normalizer such as a DNase, mannitol, hypertonic saline and/or antibiotics and/or a small molecule for treatment of CF, such as potentiator compounds for example Kalydeco (ivacaftor; VX-770), or corrector compounds, for example VX-809 (lumacaftor) and/or VX-661.

To increase access to the target cells, Broncheo-Alveolar Lavage (BAL) could be applied to clean the lungs before administration of the oligonucleotides according to the invention.

General

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

The word “substantially” does not exclude “completely” e.g. a composition which is “substantially free” from Y may be completely free from Y. Where relevant, the word “substantially” may be omitted from the definition of the invention.

The term “downstream” in relation to a nucleic acid sequence means further along the sequence in the 3′ direction; the term “upstream” means the converse. Thus in any sequence encoding a polypeptide, the start codon is upstream of the stop codon in the sense strand, but is downstream of the stop codon in the antisense strand.

References to “hybridisation” typically refer to specific hybridisation, and exclude non-specific hybridisation. Specific hybridisation can occur under experimental conditions chosen, using techniques well known in the art, to ensure that the majority of stable interactions between probe and target are where the probe and target have at least 90% sequence identity. The hybridisation conditions can be used to aid the design of probes in arrays, such that probe sequences are not used if they have more than 90% identity to other areas of the genome being analysed, to minimise cross-hybridisation. The stability of any particular probe/target duplex depend on the buffer/washing conditions used. Stable duplexes are those that remain hybridised after washing such that they will contribute to the signal obtained for that probe when reading the array.

### BRIEF DESCRIPTION OF THE DRAWINGS

FIG. 1: oligonucleotides used with the invention.

FIGS. 2 & 3: results from experiments 1 & 2, showing the proportion of genomic sequence reads in which the CTT triplet was detected. In FIG. 2 the dark bars are for the “normal” transfection and the light bars are for the “reverse” transfection. In FIG. 3 the dark bars show levels 72 hours after transfection and the light bars are 240 hours after transfection.

FIGS. 4 & 5 show ddPCR results from experiments 3 & 4, respectively, showing the rate of gene modification as represented by the % of copy numbers of wild-type CFTR sequences divided by the copy number of ΔF508 CFTR sequences. Labels on the x-axis match the names in FIG. 1 after omitting the ‘CFTR’ prefix and the internal ‘nt’, except that ‘CFTR 47 nt 3xPS as’ is ‘47 PS as’. The final ‘NT’ column represents a non-transfected negative control.

### MODES FOR CARRYING OUT THE INVENTION

Cystic fibrosis is an autosomal recessive disease among Caucasians, affecting 1 in every 30,000 people and caused by mutations in the CFTR gene (cystic fibrosis transmembrane conductance regulator. The CFTR gene (SEQ ID NO: 11) encodes a 1480-amino-acid protein that functions as a cAMP-mediated Cl⁻ channel which plays a crucial role in hydrating airway secretions and regulating other cellular functions, including Na⁺ transport, in respiratory epithelia.

The most prevalent cftr mutation (ΔF508) involves a loss of a trinucleotide at positions 1421-3 of the gene (CTT in the sense strand), leading to a loss of residue Phe-508 in the encoded polypeptide. In order to correct this mutation the inventors have designed oligonucleotides which can re-introduce the CTT triplet. These oligonucleotides have different lengths and include chemical modifications like phosphorothioate (PS) and locked-nucleic acid (LNA) residues, targeted to the sequences flanking the ΔF508 mutation.

Materials & Methods

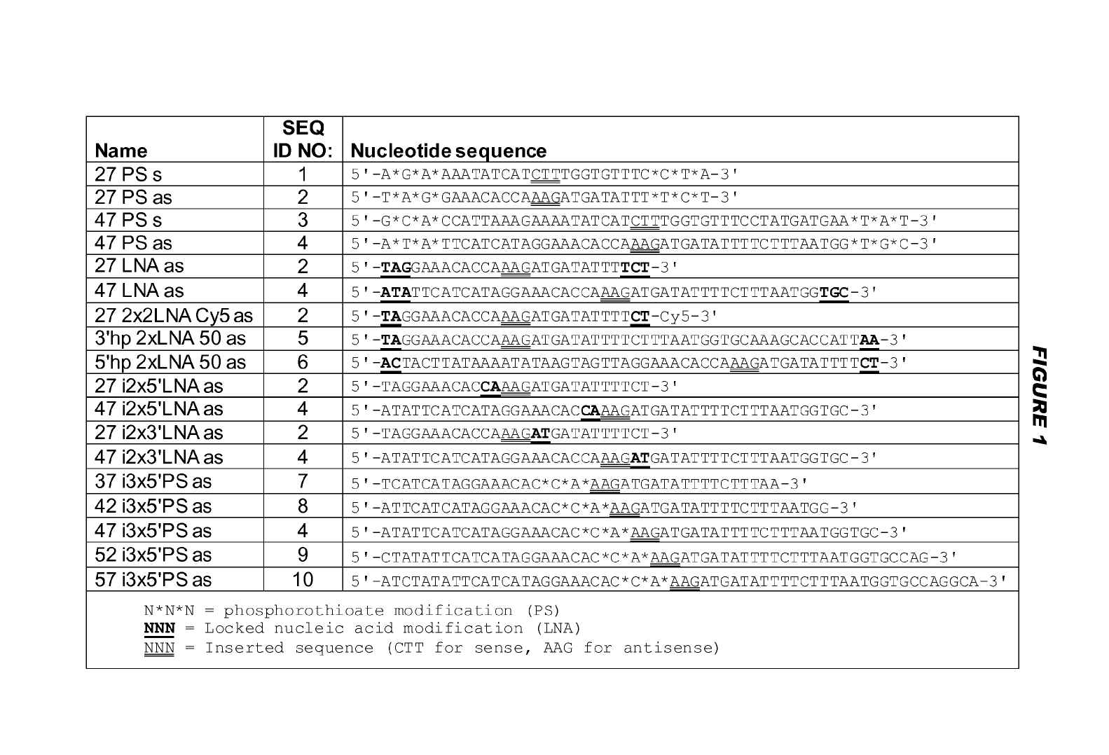

Oligonucleotides were produced and purified according manufacturer's standards (BioSpring GmbH) and reconstituted in water for injection to a final concentration of 100 μM. 15 different oligonucleotides were tested in total, as shown in FIG. 1. In some cases the oligonucleotides include a Cy5 label at the 3′ end, which was used only to facilitate detection after transfection.

CFPAC-1 cells (ATCC, CRL-1918) with the ΔF508 mutation were cultured in Dulbecco's modified Eagle's medium (Life Technologies) supplemented with 10% heat-inactivated fetal bovine serum. Cells were kept in an atmosphere of humidified air with 5% CO_{2.}

CFPAC-1 cells were transfected with the aid of the K2 transfection reagent (Biontex). In brief, 2 hours pre-transfection, cells were exposed to the K2 amplifier reagent. The oligonucleotides were diluted in 150 mM NaCl to a final volume of 50 μl. In a separate tube, 4 μl K2 transfection reagent was diluted in 46 μl 150 mM NaCl and added to the oligonucleotide mixture. After 10 second vortexing, the mix was kept at room temperature for 30 minutes before adding 50 μl to a CFPAC-1 cell suspension containing 1.0×10⁵ cells and subsequently seeded in a well of a 24-well plate. After an overnight incubation, the inoculates were replaced by fresh culture medium and incubated for 48, 72 or 240 hours at 37° C., 5% CO_{2.}

For sequencing genomic DNA, total cellular DNA from CFPAC-1 cells which had been incubated with the oligonucleotides was isolated using the NucleoSpin® Tissue XS kit (Macherey Nagel) as specified in the manufacturer's protocol, reaching a final volume of 20 μl. The recovered DNA was subjected to two different PCR protocols. In order to generate a human CFTR specific amplicon, a PCR targeting the CFTR gene was performed containing 1 μl of the isolated total cellular DNA. To this end, reactions containing 0.4 μM of forward and reverse primers, 25 μM of each dNTP, 1× AmpliTaq Gold® 360 Buffer, 3.125 mM MgCl₂ and 1.0 units of AmpliTaq Gold® 360 polymerase (all Life Technologies) were assembled. The PCR cycles were performed using the following cycling conditions. An initial denaturing step at 95° C. for 7 min was followed by 30 cycles of 30 s at 95° C., 30 s at 55° C. and 45 s at 72° C. The PCR amplifications were terminated by a final elongation period of 7 min at 72° C. 1 μl of the previous PCR was used in a nested-PCR program containing 0.4 μM of forward primers and a unique MiSeq index primer per sample, 25 μM of each dNTP, 1× AmpliTaq Gold® 360 Buffer, 3.125 mM MgCl₂ and 1.0 units AmpliTaq Gold® 360 polymerase. The PCR cycles were performed using the following cycling conditions. An initial denaturating step at 95° C. for 7 min was followed by 25 cycles of 30 s at 95° C., 30 s at 60° C. and 45 s at 72° C. Reactions were terminated using a final elongation period of 7 minutes at 72° C. In all cases the forward and reverse primers flanked the position of the triplet deletion which leads to the ΔF508 mutation.

Before loading the PCR products containing the MiSeq sequence primer sequences in the sequencer, the concentration of the purified PCR products was measured using a Qubit® 2.0 Fluorometer (Life Technologies) according the manufacturer's protocol. In summary, two Assay Tubes for the standards were made by making 20-fold dilutions of the 2 stock standards in working solution. For each sample, 200 μl of working solution was prepared in separate tubes, 1 μl of the PCR product was brought into this solution and mixed by vortexing for a couple of seconds. Samples were measured against the 2 standards and the concentration in ng/μl was calculated accordingly. Sequencing of the PCR products was performed on the MiSeq™ system from Illumina, which uses sequencing-by-synthesis to provide rapid high quality sequence data.

Experiment 1

CFPAC-1 cells were transfected using two different transfection methods, the reverse transfection whereby the transfection mixture is mixed by the cells while seeding the cells in wells of a 24-well plate and a regular transfection scheme inoculating pre-seeded CFPAC-1 cells. The transfection mixtures shown in Table 2 were used.

Pre-seeded CFPAC-1 cells (1.5×10⁵ cells/well in a 24-well) and the freshly seeded cells (reverse transfection, 1.5×10⁵ cells/well in a 24-well) were exposed to these mixture for 24 hours, after which the medium was replaced by fresh culture medium.

The cells were harvested 72 hours post-transfection and genomic DNA was isolated using the Tissue XS kit as discussed above. This material is amplified using PCR (see above) and then sequenced to determine the proportion of cells which have achieved repair of the of ΔF508 mutation.

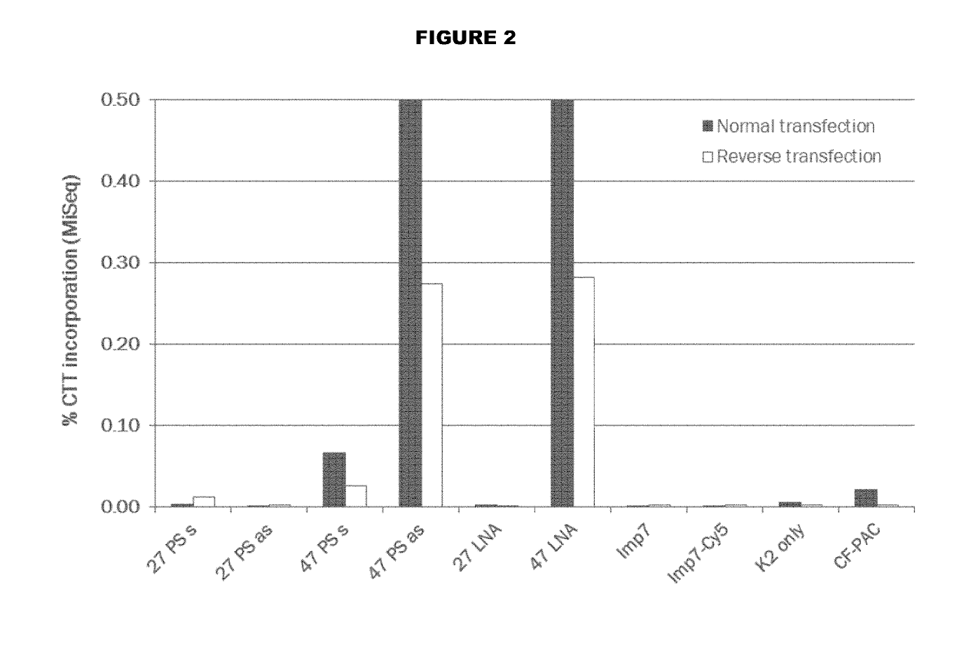

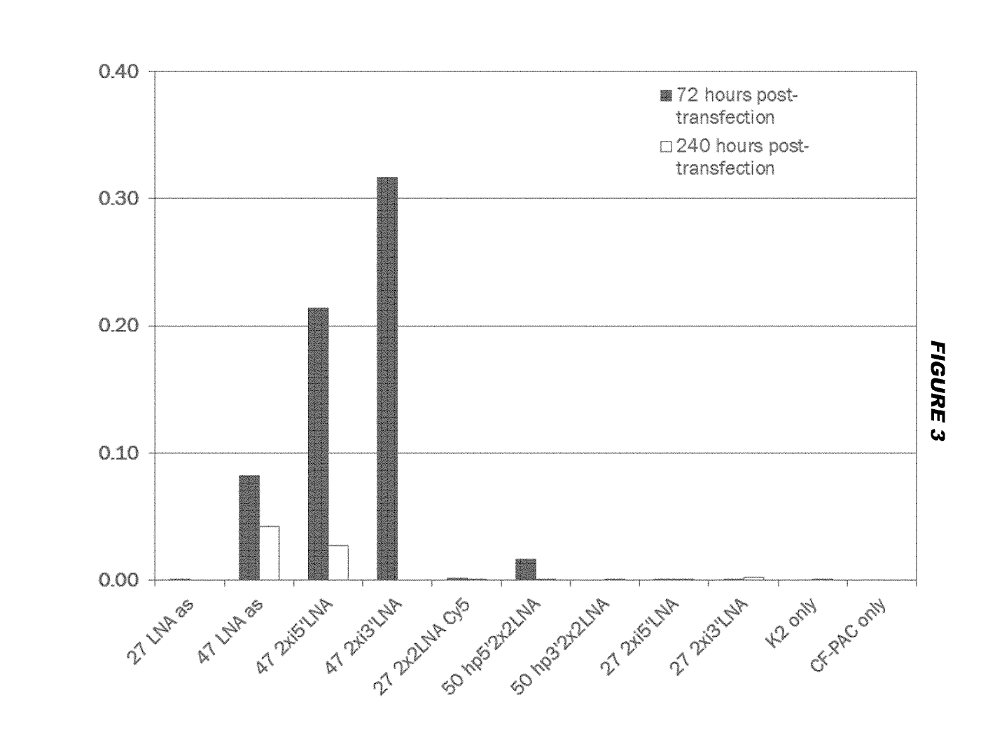

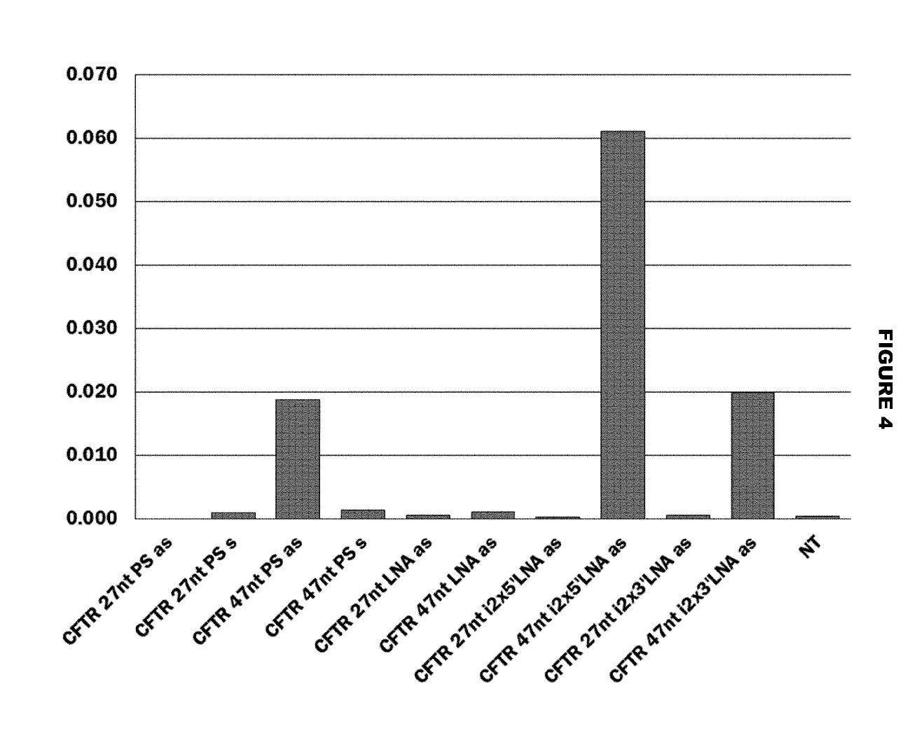

The concentrations of genomic DNA prior to PCR, and the concentrations of PCR product after amplification, are shown in Table 1. The table also shows the results of sequencing the PCR products (see also FIG. 2).

Experiment 2

As Experiment 1 had displayed an efficient transfection of CFPAC-1 cells using K2 and the reverse transfection protocol, this method was used to validate different oligonucleotides containing LNA modifications at various positions (see FIG. 1). The transfection mixtures shown in Table 4 were used.

Cells (1.5×10⁵ cells/well in a 24-well) were freshly seeded cells (reverse transfection) together with the mixtures and exposed 24 hours to the reaction mixture after which the medium was replaced by fresh culture medium. 72 or 240 hours post-transfection, the cells were harvested and genomic DNA was isolated using the Tissue XS kit, and subjected to PCR and sequencing as before. Results are in Table 3 (see also FIG. 3). Although signal had declined 10 days after transfection, this effect can be explained by dilution caused by the ongoing process of DNA integration and cell division, and even in preliminary experiments the level of repair which was seen is above background.

Experiment 3

Based on these results, we reverse-transfected CFPAC-1 cells with oligonucleotides to bring about DNA editing of the ΔF508 mutation site in CFTR. Genomic DNA of transduced cells was isolated and subjected to a droplet digital PCR (ddPCR) methodology designed to distinguish mutant and wild-type CFTR fragments. The sequence difference causes different Taqman-based probes to bind and be hydrolyzed in a 40-cycle PCR program.

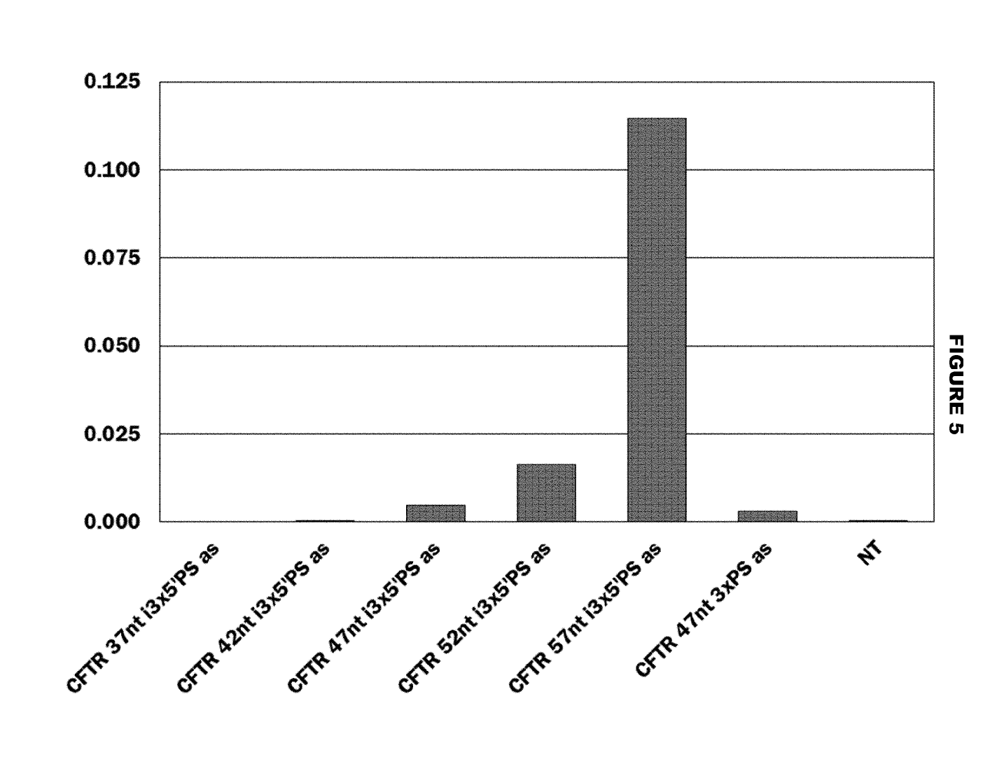

The results of this ddPCR assay are depicted in FIG. 4, and they demonstrate the ability of the different oligonucleotides to induce gene editing, introducing the missing CTT nucleotides into CFTR. As seen before, the 47 nt single-stranded antisense oligonucleotide containing phosphorothioate modification of the terminal 3 nucleotides (‘47 PS as’), as well as the sequence-identical 47 nt antisense oligonucleotide with two LNA-modified nucleotides 5′ upstream of the AAG (‘47 i2x5′LNA as’), gave the highest rate of gene conversion. Next to this, the oligonucleotide containing two LNA-modified nucleotides 3′ downstream of the AAG (‘47 i2x3′LNA as’) also gave a useful effect on gene conversion.

Experiment 4

To further investigate the gene-editing ability of 5′ internally modified oligonucleotides, we designed five further oligonucleotides with different lengths containing three PS-modified nucleotide linkages upstream of the AAG (the final 5 oligos in FIG. 1). CFPAC-1 cells were transduced (reverse transfection) and genomic DNA was isolated and subjected to the ddPCR assay. The results are shown in FIG. 5, and there is a clear length-dependent increase in modified cells as the longer oligonucleotides result in a strong increase in the percentage of wild-type CFTR (i.e. the ‘i3x5′PS as’ series, from 47-57 nt long). For comparison, ‘47 PS as’ (which showed good activity in earlier experiments) was also tested.

Clearly, the 57 nt anti-sense oligonucleotide containing three PS-modified nucleotides 5′ of the AAG outperformed the shorter versions. The enhanced rate of gene editing compared to the previously-tested ‘47 PS as’ was about 38-fold.

### CONCLUSIONS

These experiments show that the designed oligonucleotides are able to correct the ΔF508 mutation by incorporating the missing CTT sequence at the intended position in the genome of human cells.

It will be understood that the invention is described above by way of example only and modifications may be made whilst remaining within the scope and spirit of the invention.

TABLE 1

| | | Genomic | PCR | | | |
|---|---|---|---|---|---|---|
| | | DNA | product | Total | | |
| # | Sample | (ng/μl) | (ng/μl) | reads | ΔF508 | +CTT |
| 1 | 27 PS s | 167.6 | 37.2 | 475787 | 475772 | 15 |
| 2 | 27 PS as | 225.1 | 66.8 | 384348 | 384346 | 2 |
| 3 | 47 PS s | 208.7 | 56 | 453309 | 453008 | 301 |
| 4 | 47 PS as | 250.7 | 53.8 | 520887 | 503643 | 17244 |
| 5 | 27 LNA as | 381.8 | 28.2 | 524339 | 524330 | 9 |
| 6 | 47 LNA as | 641.8 | 46.8 | 473211 | 468479 | 4732 |
| 9 | K2 only | 390.0 | 41.8 | 519112 | 519084 | 28 |
| 10 | CF-PAC NT | 492.3 | 64.6 | 423495 | 423408 | 87 |
| 21 | 27 PS s | 54.4 | 57.6 | 466074 | 466021 | 53 |
| 22 | 27 PS as | 60.9 | 61.4 | 482263 | 482255 | 8 |
| 23 | 47 PS s | 29.2 | 57.8 | 501184 | 501055 | 129 |
| 24 | 47 PS as | 67.2 | 55.4 | 455909 | 454661 | 1248 |
| 25 | 27 LNA as | 115.1 | 47.8 | 136465 | 136464 | 1 |
| 26 | 47 LNA as | 93.8 | 58.4 | 433589 | 432368 | 1221 |
| 29 | K2 only | 105.1 | 46 | 501389 | 501379 | 10 |
| 30 | CF-PAC NT | 152.6 | 40.2 | 475595 | 475588 | 7 |

#21-#30 show results for the reverse transfection protocol.

TABLE 2

| | μl | μl | μl | μl | Total | μl per |
|---|---|---|---|---|---|---|
| Name | oligo | NaCl | K2 | NaCl | μl | well |
| 27 PS s | 5.54 | 49.46 | 4.4 | 50.6 | 110.00 | 50 |
| 27 PS as | 5.54 | 49.46 | 4.4 | 50.6 | 110.00 | 50 |
| 47 PS s | 2.64 | 52.36 | 4.4 | 50.6 | 110.00 | 50 |
| 47 PS as | 2.64 | 52.36 | 4.4 | 50.6 | 110.00 | 50 |
| 27 LNA as | 5.54 | 49.46 | 4.4 | 50.6 | 110.00 | 50 |
| 47 LNA as | 2.64 | 52.36 | 4.4 | 50.6 | 110.00 | 50 |
| K2 only | — | 55.00 | 4.4 | 50.6 | 110.00 | 50 |

TABLE 3

| | | Genomic | PCR | | | |
|---|---|---|---|---|---|---|
| | | DNA | product | Total | | |
| # | Sample | (ng/μl) | (ng/μl) | reads | ΔF508 | +CTT |
| 1 | 27 LNA as | 167.6 | 290 | 434123 | 434122 | 1 |
| 2 | 47 LNA as | 225.1 | 260 | 412543 | 412203 | 340 |
| 3 | 47 2xi5′LNA | 208.7 | 293 | 402770 | 401909 | 861 |
| 4 | 47 2xi3′LNA | 250.7 | 243 | 525211 | 523547 | 1664 |
| 5 | 27 2x2LNA Cy5 | 381.8 | 213 | 448169 | 448163 | 6 |
| 6 | 50 hp5′2x2LNA | 641.8 | 255 | 404853 | 404788 | 65 |
| 7 | 50 hp3′2x2LNA | 458.1 | 266 | 495851 | 495851 | |
| 8 | 27 2xi5′LNA | 352.9 | 285 | 467278 | 467277 | 1 |
| 9 | 27 2xi3′LNA | 390.0 | 259 | 543384 | 543380 | 4 |
| 10 | K2 only | 492.3 | 283 | 403416 | 403416 | |
| 21 | 27 LNA as | 54.4 | 248 | 428907 | 428907 | |
| 22 | 47 LNA as | 60.9 | 283 | 492964 | 492755 | 209 |
| 23 | 47 2xi5′LNA | 29.2 | 253 | 403734 | 403625 | 109 |
| 24 | 47 2xi3′LNA | 67.2 | 263 | 206 | 205 | 1 |
| 25 | 27 2x2LNA Cy5 | 115.1 | 243 | 463985 | 463982 | 3 |
| 26 | 50 hp5′2x2LNA | 93.8 | 270 | 348200 | 348198 | 2 |
| 27 | 50 hp3′2x2LNA | 29.0 | 283 | 285860 | 285858 | 2 |
| 28 | 27 2xi5′LNA | 26.7 | 281 | 413794 | 413793 | 1 |
| 29 | 27 2xi3′LNA | 105.1 | 299 | 465890 | 465881 | 9 |
| 30 | K2 only | 152.6 | 230 | 489875 | 489870 | 5 |

#21-#30 show results 240 hours after transfection.

TABLE 4

| | μl | μl | μl | μl | Total | μl per |
|---|---|---|---|---|---|---|
| Name | oligo | NaCl | K2 | NaCl | μl | well |
| 27 LNA as | 5.54 | 49.46 | 4.4 | 50.6 | 110.00 | 50 |
| 47 LNA as | 2.64 | 52.36 | 4.4 | 50.6 | 110.00 | 50 |
| 47 i2x5′LNA as | 2.64 | 52.36 | 4.4 | 50.6 | 110.00 | 50 |
| 47 i2x3′LNA as | 2.64 | 52.36 | 4.4 | 50.6 | 110.00 | 50 |
| 27 2x2LNACy5 as | 5.54 | 49.46 | 4.4 | 50.6 | 110.00 | 50 |
| 3′hp2xLNA50 as | 2.64 | 52.36 | 4.4 | 50.6 | 110.00 | 50 |
| 5′hp2xLNA50 as | 2.64 | 52.36 | 4.4 | 50.6 | 110.00 | 50 |
| 27nti2x5′LNA as | 5.54 | 49.46 | 4.4 | 50.6 | 110.00 | 50 |
| 27nti2x3′LNA as | 5.54 | 49.46 | 4.4 | 50.6 | 110.00 | 50 |
| K2 only | — | 55.00 | 4.4 | 50.6 | 110.00 | 50 |

SEQUENCE LISTING
(F508 codon position underlined)

SEQ ID NO: 1
5′-AGAAAATATCATCTTTGGTGTTTCCTA-3′
SEQ ID NO: 2
5′-TAGGAAACACCAAAGATGATATTTTCT-3′
SEQ ID NO: 3
5′-GCACCATTAAAGAAAATATCATCTTTGGTGTTTCCTATGATGAAT
AT-3′
SEQ ID NO: 4
5′-ATATTCATCATAGGAAACACCAAAGATGATATTTTCTTTAATGGT
GC-3′
SEQ ID NO: 5
5′-TAGGAAACACCAAAGATGATATTTTCTTTAATGGTGCAAAGCACC
ATTAA-3′
SEQ ID NO: 6
5′-ACTACTTATAAAATATAAGTAGTTAGGAAACACCAAAGATGATAT
TTTCT-3′
SEQ ID NO: 7
5′-TCATCATAGGAAACACCAAAGATGATATTTTCTTTAA-3′
SEQ ID NO: 8
5′-ATTCATCATAGGAAACACCAAAGATGATATTTTCTTTAATGG-3′
SEQ ID NO: 9
5′-CTATATTCATCATAGGAAACACCAAAGATGATATTTTCTTTAATG
GTGCCAG-3′
SEQ ID NO: 10
5′-ATCTATATTCATCATAGGAAACACCAAAGATGATATTTTCTTTAA
TGGTGCCAGGCA-3′
SEQ ID NO: 11
ATGCAGAGGTCGCCTCTGGAAAAGGCCAGCGTTGTCTCCAAACTTTTT
TTCAGCTGGACCAGACCAATTTTGAGGAAAGGATACAGACAGCGCCTG
GAATTGTCAGACATATACCAAATCCCTTCTGTTGATTCTGCTGACAAT
CTATCTGAAAAATTGGAAAGAGAATGGGATAGAGAGCTGGCTTCAAAG
AAAAATCCTAAACTCATTAATGCCCTTCGGCGATGTTTTTTCTGGAGA
TTTATGTTCTATGGAATCTUTTATATTTAGGGGAAGTCACCAAAGCAG
TACAGCCTCTCTTACTGGGAAGAATCATAGCTTCCTATGACCCGGATA
ACAAGGAGGAACGCTCTATCGCGATTTATCTAGGCATAGGCTTATGCC
TTCTCTTTATTGTGAGGACACTGCTCCTACACCCAGCCATTTTTGGCC
TTCATCACATTGGAATGCAGATGAGAATAGCTATGTTTAGTTTGATTT
ATAAGAAGACTTTAAAGCTGTCAAGCCGTGTTCTAGATAAAATAAGTA
TTGGACAACTTGTTAGTCTCCTTTCCAACAACCTGAACAAATTTGATG
AAGGACTTGCATTGGCACATTTCGTGTGGATCGCTCCTTTGCAAGTGG
CACTCCTCATGGGGCTAATCTGGGAGTTGTTACAGGCGTCTGCCTTCT
GTGGACTTGGTTTCCTGATAGTCCTTGCCCTTTTTCAGGCTGGGCTAG
GGAGAATGATGATGAAGTACAGAGATCAGAGAGCTGGGAAGATCAGTG
AAAGACTTGTGATTACCTCAGAAATGATTGAAAATATCCAATCTGTTA
AGGCATACTGCTGGGAAGAAGCAATGGAAAAAATGATTGAAAACTTAA
GACAAACAGAACTGAAACTGACTCGGAAGGCAGCCTATGTGAGATACT
TCAATAGCTCAGCCTTCTTCTTCTCAGGGTTCTTTGTGGTGTTTTTAT
CTGTGCTTCCCTATGCACTAATCAAAGGAATCATCCTCCGGAAAATAT
TCACCACCATCTCATTCTGCATTGTTCTGCGCATGGCGGTCACTCGGC
AATTTCCCTGGGCTGTACAAACATGGTATGACTCTCTTGGAGCAATAA
ACAAAATACAGGATTTCTTACAAAAGCAAGAATATAAGACATTGGAAT
ATAACTTAACGACTACAGAAGTAGTGATGGAGAATGTAACAGCCTTCT
GGGAGGAGGGATTTGGGGAATTATTTGAGAAAGCAAAACAAAACAATA
ACAATAGAAAAACTTCTAATGGTGATGACAGCCTCTTCTTCAGTAATT
TCTCACTTCTTGGTACTCCTGTCCTGAAAGATATTAATTTCAAGATAG
AAAGAGGACAGTTGTTGGCGGTTGCTGGATCCACTGGAGCAGGCAAGA
CTTCACTTCTAATGATGATTATGGGAGAACTGGAGCCTTCAGAGGGTA
AAATTAAGCACAGTGGAAGAATTTCATTCTGTTCTCAGTTTTCCTGGA
TTATGCCTGGCACCATTAAAGAAAATATCATCTTTGGTGTTTCCTATG
ATGAATATAGATACAGAAGCGTCATCAAAGCATGCCAACTAGAAGAGG
ACATCTCCAAGTTTGCAGAGAAAGACAATATAGTTCTTGGAGAAGGTG
GAATCACACTGAGTGGAGGTCAACGAGCAAGAATTTCTTTAGCAAGAG
CAGTATACAAAGATGCTGATTTGTATTTATTAGACTCTCCTTTTGGAT
ACCTAGATGTTTTAACAGAAAAAGAAATATTTGAAAGCTGTGTCTGTA
AACTGATGGCTAACAAAACTAGGATTTTGGTCACTTCTAAAATGGAAC
ATTTAAAGAAAGCTGACAAAATATTAATTTTGCATGAAGGTAGCAGCT
ATTTTTATGGGACATTTTCAGAACTCCAAAATCTACAGCCAGACTTTA
GCTCAAAACTCATGGGATGTGATTCTTTCGACCAATTTAGTGCAGAAA
GAAGAAATTCAATCCTAACTGAGACCTTACACCGTTTCTCATTAGAAG
GAGATGCTCCTGTCTCCTGGACAGAAACAAAAAAACAATCTTTTAAAC
AGACTGGAGAGTTTGGGGAAAAAAGGAAGAATTCTATTCTCAATCCAA
TCAACTCTATACGAAAATTTTCCATTGTGCAAAAGACTCCCTTACAAA
TGAATGGCATCGAAGAGGATTCTGATGAGCCTTTAGAGAGAAGGCTGT
CCTTAGTACCAGATTCTGAGCAGGGAGAGGCGATACTGCCTCGCATCA
GCGTGATCAGCACTGGCCCCACGCTTCAGGCACGAAGGAGGCAGTCTG
TCCTGAACCTGATGACACACTCAGTTAACCAAGGTCAGAACATTCACC
GAAAGACAACAGCATCCACACGAAAAGTGTCACTGGCCCCTCAGGCAA
ACTTGACTGAACTGGATATATATTCAAGAAGGTTATCTCAAGAAACTG
GCTTGGAAATAAGTGAAGAAATTAACGAAGAAGACTTAAAGGAGTGCT
TTTTTGATGATATGGAGAGCATACCAGCAGTGACTACATGGAACACAT
ACCTTCGATATATTACTGTCCACAAGAGCTTAATTTTTGTGCTAATTT
GGTGCTTAGTAATTTTTCTGGCAGAGGTGGCTGCTTCTTTGGTTGTGC
TGTGGCTCCTTGGAAACACTCCTCTTCAAGACAAAGGGAATAGTACTC
ATAGTAGAAATAACAGCTATGCAGTGATTATCACCAGCACCAGTTCGT
ATTATGTGUTTACATTTACGTGGGAGTAGCCGACACTTTGCTTGCTAT
GGGATTCTTCAGAGGTCTACCACTGGTGCATACTCTAATCACAGTGTC
GAAAATTTTACACCACAAAATGTTACATTCTGTTCTTCAAGCACCTAT
GTCAACCCTCAACACGTTGAAAGCAGGTGGGATTCTTAATAGATTCTC
CAAAGATATAGCAATTTTGGATGACCTTCTGCCTCTTACCATATTTGA
CTTCATCCAGTTGTTATTAATTGTGATTGGAGCTATAGCAGTTGTCGC
AGTTTTACAACCCTACATCTTTGTTGCAACAGTGCCAGTGATAGTGGC
TTTTATTATGTTGAGAGCATATTTCCTCCAAACCTCACAGCAACTCAA
ACAACTGGAATCTGAAGGCAGGAGTCCAATTTTCACTCATCTTGTTAC
AAGCTTAAAAGGACTATGGACACTTCGTGCCTTCGGACGGCAGCCTTA
CTTTGAAACTCTGTTCCACAAAGCTCTGAATTTACATACTGCCAACTG
GTTCTTGTACCTGTCAACACTGCGCTGGTTCCAAATGAGAATAGAAAT
GATTTTTGTCATCTTCTTCATTGCTGTTACCTTCATTTCCATTTTAAC
AACAGGAGAAGGAGAAGGAAGAGTTGGTATTATCCTGACTTTAGCCAT
GAATATCATGAGTACATTGCAGTGGGCTGTAAACTCCAGCATAGATGT
GGATAGCTTGATGCGATCTGTGAGCCGAGTCTTTAAGTTCATTGACAT
GCCAACAGAAGGTAAACCTACCAAGTCAACCAAACCATACAAGAATGG
CCAACTCTCGAAAGTTATGATTATTGAGAATTCACACGTGAAGAAAGA
TGACATCTGGCCCTCAGGGGGCCAAATGACTGTCAAAGATCTCACAGC
AAAATACACAGAAGGTGGAAATGCCATATTAGAGAACATTTCCTTCTC
AATAAGTCCTGGCCAGAGGGTGGGCCTCTTGGGAAGAACTGGATCAGG
GAAGAGTACTTTGTTATCAGCTTTTTTGAGACTACTGAACACTGAAGG
AGAAATCCAGATCGATGGTGTGTCTTGGGATTCAATAACTTTGCAACA
GTGGAGGAAAGCCTTTGGAGTGATACCACAGAAAGTATTTATTTTTTC
TGGAACATTTAGAAAAAACTTGGATCCCTATGAACAGTGGAGTGATCA
AGAAATATGGAAAGTTGCAGATGAGGTTGGGCTCAGATCTGTGATAGA
ACAGTTTCCTGGGAAGCTTGACTTTGTCCTTGTGGATGGGGGCTGTGT
CCTAAGCCATGGCCACAAGCAGTTGATGTGCTTGGCTAGATCTGTTCT
CAGTAAGGCGAAGATCTTGCTGCTTGATGAACCCAGTGCTCATTTGGA
TCCAGTAACATACCAAATAATTAGAAGAACTCTAAAACAAGCATTTGC
TGATTGCACAGTAATTCTCTGTGAACACAGGATAGAAGCAATGCTGGA
ATGCCAACAATTTTTGGTCATAGAAGAGAACAAAGTGCGGCAGTACGA
TTCCATCCAGAAACTGCTGAACGAGAGGAGCCTCTTCCGGCAAGCCAT
CAGCCCCTCCGACAGGGTGAAGCTCTTTCCCCACCGGAACTCAAGCAA
GTGCAAGTCTAAGCCCCAGATTGCTGCTCTGAAAGAGGAGACAGAAGA
AGAGGTGCAAGATACAAGGCTT

1. A method for making a change in an endogenous chromosomal DNA sequence of a mammalian cell, comprising steps of: (i) introducing into said cell an oligonucleotide having a sequence that is complementary to the chromosomal DNA sequence except for the change; (ii) allowing sufficient time for the cell to incorporate the change into the endogenous chromosomal DNA sequence through endogenous nucleic acid modifying pathways; and (iii) identifying the presence of the change in the chromosomal DNA sequence.

2. A method according to claim 1, wherein the target sequence in the chromosome is a sequence comprising a mutation in the CFTR gene.

3. A method for making a change in an endogenous mutant CFTR chromosomal DNA sequence of a human cell, comprising steps of: (i) introducing into said cell an oligonucleotide having a sequence that is complementary to the chromosomal DNA sequence except for the change; and (ii) allowing sufficient time for the cell to incorporate the change into the endogenous chromosomal DNA sequence through endogenous nucleic acid modifying pathways.

4. A method according to any preceding claim, wherein the cell is a human cell.

5. A method according to claim 4, wherein the cell is a pluripotent stem cell, a cell residing in an organoid, or a cell residing in an entire organism.

6. A method according to any preceding claim, wherein the change is an insertion of one or more nucleotides (e.g. up to 6 nucleotides) into the endogenous chromosomal DNA sequence.

7. A method according to any one of claims 2 to 6, wherein the endogenous chromosomal DNA sequence is CFTR gene which encodes a polypeptide with deletion of phenylalanine in position 508 (ΔF508) and the change comprises an insertion of three nucleotides to insert an amino acid at position 508 and thereby restore a functional CFTR polypeptide.

8. A method according to any preceding claim, wherein the oligonucleotide is between 20 and 80 nucleotides in length, preferably between 20 and 50 or 25 and 75 nucleotides.

9. A method according to any preceding claim, wherein the oligonucleotide comprises 2′-deoxynucleotide residues, optionally comprising chemical modifications of its sugar moieties, purines, pyrimidines or backbone.

10. A method according to claim 9, wherein the oligonucleotide comprises 2′-deoxynucleotides with one or more phosphorothioate (PS-) linkages and/or one or more locked nucleosides.

11. A method according to any preceding claim, wherein the oligonucleotide is complementary to the sense strand of the endogenous chromosomal DNA sequence.

12. A method according to any preceding claim, utilising an oligonucleotide as defined in any one of claims 16 to 25.

13. A method according to any preceding claim, wherein said cell is a lung cell residing in a human subject and said oligonucleotide is administered to the lung of the subject through inhalation.

14. A method according to claim 13, wherein the oligonucleotide is formulated in iso- or hypotonic saline.

15. The method of any preceding claim, utilising an oligonucleotide as defined in any one of claims 16-33.

16. An oligonucleotide for making a desired insertion or substitution at a specific position in a chosen strand of a target chromosomal DNA sequence, having sequence 5′-X-Y-Z-3′, wherein: X is complementary to the chromosomal sequence downstream in the non-chosen strand of the specific position; Z is complementary to the chromosomal sequence upstream in the non-chosen strand of the specific position; and Y is the desired insertion or substitution; and wherein (i) X and/or Z is/are linked to Y by a phosphorothioate linkage, and/or (ii) the 3′ nucleotide of X and/or the 5′ nucleotide of Z is a locked nucleotide.

17. An oligonucleotide for making a desired insertion or substitution at a specific position in a chosen strand of a target chromosomal DNA sequence, having sequence 5′-X-Y-Z-3′, wherein: X is complementary to the chromosomal sequence downstream in the non-chosen strand of the specific position; Z is complementary to the chromosomal sequence upstream in the non-chosen strand of the specific position; and Y is the desired insertion or substitution.

18. The oligonucleotide of claim 16 or claim 17, wherein the chosen strand is the antisense strand and the non-chosen strand is the sense strand.

19. An oligonucleotide having a sequence that is complementary to a target sequence in an endogenous mammalian chromosomal DNA sequence, except that it includes at an internal position a desired modification of the target sequence, wherein the nucleotide immediately upstream of the internal position is a locked nucleotide.

20. An oligonucleotide having a sequence that is complementary to a target sequence in an endogenous mammalian chromosomal DNA sequence, except that it includes a desired modification of the target sequence, wherein (i) at least the 5′ and/or 3′ terminal nucleotides of the oligonucleotide is/are locked; and/or (ii) at least the 5′ and/or 3′ terminal dinucleotides of the oligonucleotide are linked via a phosphorothioate linkage.

21. An oligonucleotide for restoring function to CFTR having a ΔF508 mutation, having sequence 5′-X-Y-Z-3′, wherein:
X is complementary to the sense strand of the human CFTR gene, starting at nucleotide 1524; Z is complementary to the sense strand of the human CFTR gene, up to nucleotide 1520; and Y is a trinucleotide AAG, AAA, AAT, CCG, CAG, CCA, CAA, CCT or CAT; or
X is complementary to the antisense strand of the human CFTR gene, up to nucleotide 1520; Z is complementary to the antisense strand of the human CFTR gene, starting at nucleotide 1524; and Y is a trinucleotide CTT, TTT, ATT, CGG, CTG, TGG, TTG, AGG or ATG; or
X is complementary to the sense strand of the human CFTR gene, starting at nucleotide 1525; Z is complementary to the sense strand of the human CFTR gene, up to nucleotide 1521; and Y is a trinucleotide AAA, GAA, CCC, TCC, GCC, ACC, ACA, GCA, or CAT; or
X is complementary to the antisense strand of the human CFTR gene, up to nucleotide 1521; Z is complementary to the antisense strand of the human CFTR gene, starting at nucleotide 1525; and Y is a trinucleotide TTT, TTC, GGT, GGC, GGA, GGG, TGT, TGC, or ATG.

22. The oligonucleotide of any one of claims 17-21, including at least one non-naturally occurring nucleotide.

23. The oligonucleotide of claim 22, including one or more locked nucleoside(s) and/or one or more phosphorothioate internucleotide linkage(s).

24. The oligonucleotide of claim 23, wherein at least two neighbouring nucleotides are both locked.

25. The oligonucleotide of claim 23, wherein the at least two neighbouring nucleotides are (a) at the 5′ end of the oligonucleotide, (b) at the 3′ end of the oligonucleotide, (c) immediately to the 5′ of the portion of the oligonucleotide which specifies the desired modification of the target sequence, or (d) immediately to the 3′ of the portion of the oligonucleotide which specifies the desired modification of the target sequence.

26. An oligonucleotide comprising the nucleotide sequence of any one of SEQ ID NOs: 1-6 or of any one of SEQ ID NOs: 7-10.

27. An oligonucleotide comprising the nucleotide sequence of SEQ ID NO: 4.

28. The oligonucleotide of claim 27, comprising one or more phosphorothioate (PS-) linkages and/or one or more locked nucleotides.

29. The oligonucleotide of claim 28, comprising one or more (e.g. two or three) consecutive PS-linkages directly upstream or downstream of the AAG trinucleotide at positions 23-25 of SEQ ID NO: 4.

30. The oligonucleotide of any one of claims 26-28, which is an oligodeoxynucleotide.

31. The oligonucleotide of any one of claims 26-29, which is 27-80 nucleotides long.

32. The oligonucleotide of any one of claims 26-30, modified as follows:

| SEQ | |
|---|---|
| ID | |
| NO: | Modifications |
| 3 | 6 PS linkages, between the 4 terminal nucleotides at both ends |
| 4 | 6 PS linkages, between the 4 terminal nucleotides at both ends |
| 4 | 6 locked nucleotides: 3 at each end |
| 4 | 2 locked nucleotides (nucleotides 21 & 22) |
| 4 | 2 locked nucleotides (nucleotides 26 & 27) |
| 4 | 3 PS linkages, between nucleotides 20-21, 21-22, and 22-23 |
| 9 | 3 PS linkages, between nucleotides 22-23, 23-24, and 24-25 |
| 10 | 3 PS linkages, between nucleotides 24-25, 25-26, and 26-27 |

33. The oligonucleotide of any one of claims 26-31, as shown in FIG. 1.

34. An oligonucleotide according to any one of claims 16-32, formulated in isotonic or hypotonic saline.

35. The oligonucleotide according to any one of claims 16-32, for use in the method of any one of claims 1 to 14.

36. An oligonucleotide sequence for correcting a mutation in a target sequence of a chromosome in a target cell of a mammalian, preferably human, subject, wherein the oligonucleotide is complementary to the target sequence except for the corrected sequence, said oligonucleotide being in a form ready for uptake by said the target cells.


SEQUENCE LISTING (11 sequences)

SEQ ID NO: 1 - DNA, 27 bp, Artificial Sequence
  Antisense oligonucleotide
agaaaatatcatctttggtgtttccta

SEQ ID NO: 2 - DNA, 27 bp, Artificial Sequence
  Antisense oligonucleotide
taggaaacaccaaagatgatattttct

SEQ ID NO: 3 - DNA, 47 bp, Artificial Sequence
  Antisense oligonucleotide
gcaccattaaagaaaatatcatctttggtgtttcctatgatgaatat

SEQ ID NO: 4 - DNA, 47 bp, Artificial Sequence
  Antisense oligonucleotide
atattcatcataggaaacaccaaagatgatattttctttaatggtgc

SEQ ID NO: 5 - DNA, 50 bp, Artificial Sequence
  Antisense oligonucleotide
taggaaacaccaaagatgatattttctttaatggtgcaaagcaccattaa

SEQ ID NO: 6 - DNA, 50 bp, Artificial Sequence
  Antisense oligonucleotide
actacttataaaatataagtagttaggaaacaccaaagatgatattttct

SEQ ID NO: 7 - DNA, 37 bp, Artificial Sequence
  Antisense oligonucleotide
tcatcataggaaacaccaaagatgatattttctttaa

SEQ ID NO: 8 - DNA, 42 bp, Artificial Sequence
  Antisense oligonucleotide
attcatcataggaaacaccaaagatgatattttctttaatgg

SEQ ID NO: 9 - DNA, 52 bp, Artificial Sequence
  Antisense oligonucleotide
ctatattcatcataggaaacaccaaagatgatattttctttaatggtgccag

SEQ ID NO: 10 - DNA, 57 bp, Artificial Sequence
  Antisense oligonucleotide
atctatattcatcataggaaacaccaaagatgatattttctttaatggtgccaggca

SEQ ID NO: 11 - DNA, 4440 bp, Homo sapiens
atgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctt